(19) United States
(12) Patent Application Publication
(10) Pub. No.: US 2013/0157322 A1
(43) Pub. Date: Jun. 20, 2013

(54) PROCESS FOR PRODUCING ETHANOL AND ETHYLENE VIA FERMENTATION

(71) Applicants: Sean Dennis Simpson, Auckland (NZ); Christophe Daniel Mihalcea, Auckland (NZ)

(72) Inventors: Sean Dennis Simpson, Auckland (NZ); Christophe Daniel Mihalcea, Auckland (NZ)

(73) Assignee: LANZATECH NEW ZEALAND LIMITED, Roselle, IL (US)

(21) Appl. No.: 13/817,720

(22) PCT Filed: Aug. 26, 2011
(86) PCT No.: PCT/NZ11/00170

§ 371 Date: Feb. 19, 2013

Related U.S. Application Data
(60) Provisional application No. 61/377,309, filed on Aug. 26, 2010.

(51) Int. Cl. C07C 1/24

(52) U.S. Cl. CPC C07C 1/24
USPC 435/123; 435/166; 435/167

(57) ABSTRACT

A process for converting a substrate such as carbon monoxide to useful chemicals has been developed. The process involves providing a substrate comprising CO to a bioreactor which contains a culture of one or more micro-organisms and anaerobically fermenting the substrate to produce ethanol. The ethanol is next converted to one or more chemical products via the compound ethylene. The source of the CO can be an industrial process such as the ferrous metal products manufacturing. The microorganism can be Clostridium autoethanogenum, Clostridium ljundahlii or Clostridium ragsdalei.

### FIELD

The present invention relates to the production of one or more chemical products utilising a step involving microbial fermentation, particularly microbial fermentation of substrates comprising CO.

### BACKGROUND

Ethylene is a high value gaseous compound which is widely used in industry. By way of example, ethylene may be used as an anaesthetic or as a fruit ripening agent, as well as in the production of a number of other chemical products. By way of example, ethylene may be used to produce polyethylene, ethylene oxide, ethylene dichloride, ethylene dibromide, ethyl chloride and ethylbenzene, which in turn can be used to produce other useful downstream products.

Carbon Monoxide (CO) is a major by-product of the incomplete combustion of organic materials such as coal or oil and oil derived products. Although the complete combustion of carbon containing precursors yields CO2 and water as the only end products, some industrial processes need elevated temperatures favouring the build up of carbon monoxide over CO2. One example is the steel industry, where high temperatures are needed to generate desired steel qualities. For example, the steel industry in Australia is reported to produce and release into the atmosphere over 500,000 tonnes of CO annually.

Furthermore, CO is also a major component of syngas, where varying amounts of CO and H2 are generated by gasification of a carbon-containing fuel. For example, syngas may be produced by cracking the organic biomass of waste woods and timber to generate precursors for the production of fuels and more complex chemicals.

The release of CO into the atmosphere may have significant environmental impact. In addition, emissions taxes may be required to be paid, increasing costs to industrial plants.

Since CO is a reactive energy rich molecule, it can be used as a precursor compound for the production of a variety of chemicals.

### OBJECT

It is an object of the present invention to provide a process for the production of one or more chemical products, including a process which produces ethylene, that overcomes or ameliorates one or more of the disadvantages of the prior art, or to at least to provide the public with a useful choice.

### STATEMENT OF INVENTION

In one aspect, the invention provides a method of producing one or more chemical products the method comprising at least the step of anaerobically fermenting a substrate comprising CO to produce ethanol.

In one embodiment, the method comprises at least:
a. anaerobically fermenting a substrate comprising CO to ethanol; and,
b. converting the ethanol to one or more chemical products via the intermediate compound ethylene.

In one embodiment, the method comprises recovering the ethanol after step a. before it is converted to ethylene or one or more chemical products in step b.

In one embodiment, the method comprises recovering ethylene during step b. In another embodiment, ethanol is converted to one or more chemical products without recovery of ethylene during step b.

In one embodiment, step a. comprises providing a substrate comprising CO and in a bioreactor containing a culture of one or more micro-organisms, anaerobically fermenting the substrate to produce ethanol.

In one embodiment, ethanol is converted to one or more chemical products by one or more chemical processes. In one embodiment, the ethanol is converted to one or more chemical products by one or more chemical processes including one or more chemical synthesis steps.

In one aspect, the invention provides a method of producing ethylene the method comprising at least the step of anaerobically fermenting a substrate comprising CO to produce ethanol.

In one embodiment, the method comprises at least:
a. anaerobically fermenting a substrate comprising CO to ethanol; and,
b. converting the ethanol ethylene.

In one embodiment, the method comprises recovering the ethanol after step a. before it is converted to ethylene in step b.

In one embodiment, the method comprises recovering ethylene during or after step b. In one embodiment, the method further comprises converting or using ethylene in the production of one or more chemical products following recovery of ethylene.

In another embodiment, ethanol is converted to one or more chemical products without recovery of ethylene from the method.

In one embodiment, step a. comprises providing a substrate comprising CO and in a bioreactor containing a culture of one or more micro-organisms, anaerobically fermenting the substrate to produce ethanol.

In particular embodiments of the various aspects, the substrate comprising carbon monoxide is a gaseous substrate comprising carbon monoxide. The gaseous substrate comprising carbon monoxide can be obtained as a by-product of an industrial process. In certain embodiments, the industrial process is selected from the group consisting of ferrous metal products manufacturing, non-ferrous products manufacturing, petroleum refining processes, gasification of biomass, gasification of coal, electric power production, carbon black production, ammonia production, methanol production and coke manufacturing. In one embodiment the gaseous substrate comprises a gas obtained from a steel mill. In another embodiment ‘the gaseous substrate comprises automobile exhaust fumes.

In particular embodiments, the CO-containing substrate typically contains a major proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

In certain embodiments of the various aspects, the method comprises microbial fermentation using a microorganism of the genus Clostridia.

In one embodiment, the method comprises microbial fermentation using Clostridium autoethanogenum.

In one embodiment, the method comprises microbial fermentation using Clostridium Ijundahlii.

In one embodiment, the method comprises microbial fermentation using Clostridium ragsdalei.

In one embodiment the ethanol is converted to ethylene by chemical synthesis.

In one embodiment, the methods of the invention are continuous. In certain embodiments ethanol is continuously recovered from the bioreactor. In certain embodiments, the ethanol recovered from the bioreactor is fed directly for conversion to ethylene.

In another aspect, the invention provides ethylene produced by a method as herein before described.

In another aspect, the invention provides one or more chemical products produced by a method as herein before described.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE DRAWINGS

The invention will now be described in detail with reference to the accompanying Figures in which:

FIG. 1: Shows the ethanol production of DSM19630 (FIG. 1a) and DSM23693 (FIG. 1b)

FIG. 2: Shows the ethanol production of C. autoethanogenum, C ljungdahlii and C.ragsdalei.

### DETAILED DESCRIPTION OF THE INVENTION

The following is a description of the present invention, including preferred embodiments thereof, given in general terms. The invention is further exemplified in the disclosure given under the heading “Examples” herein below, which provides experimental data supporting the invention, specific examples of aspects of the invention, and means of performing the invention.

The phrase “one or more chemical products” is used herein to refer to chemical compounds or products which can be manufactured from or using ethylene, and includes products in which ethylene are considered intermediates in the production of. Various non-limiting examples of such chemical products are provided herein after.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. As is described herein after, in some embodiments the bioreactor may comprise a first growth reactor and a second fermentation reactor. As such, when referring to the addition of a substrate, for example a substrate comprising carbon monoxide, to the bioreactor or fermentation reaction it should be understood to include addition to either or both of these reactors where appropriate.

The term “substrate comprising carbon monoxide” and like terms should be understood to include any substrate in which carbon monoxide is available to one or more strains of bacteria for growth and/or fermentation, for example.

“Gaseous substrates comprising carbon monoxide” include any gas which contains a level of carbon monoxide. The gaseous substrate will typically contain a major proportion of CO, preferably at least about 15% to about 95% CO by volume.

Unless the context requires otherwise, the phrases “fermenting”, “fermentation process” or “fermentation reaction” and the like, as used herein, are intended to encompass both the growth phase and product biosynthesis phase of the process.

In one aspect, the invention provides a method of producing one or more chemical products the method comprising at least the step of anaerobically fermenting a substrate comprising CO to produce ethanol. In one embodiment, the method comprises at least anaerobically fermenting a substrate comprising CO to produce ethanol and converting the ethanol to one or more chemical products via the intermediate compound ethylene.

In another aspect, the invention provides a method of producing ethylene, the method comprising as least anaerobically fermenting a substrate comprising CO to produce ethanol. In one embodiment, the method comprises at least anaerobically fermenting a substrate comprising CO to produce ethanol and then converting the ethanol to ethylene.

In one embodiment, the methods of the invention comprise recovering the ethylene from the fermentation broth before it is converted to ethylene. However, in some embodiments, this may not be necessary.

In one embodiment, the methods comprise recovering ethylene produced and following recovery converting or using it in the production of one or more chemical products. In other embodiments, it is not necessary to recover ethylene before it is converted or used to produce one or more chemical products.

In one embodiment, the microbial fermentation comprises providing a substrate comprising CO and in a bioreactor containing a culture of one or more micro-organisms, anaerobically fermenting the substrate to produce ethanol.

In certain embodiments, the methods of the invention are continuous. In one embodiment ethanol is continuously recovered from the fermentation broth or bioreactor. In certain embodiments, the ethanol recovered from the fermentation broth or bioreactor is fed directly for chemical conversion to ethylene. For example, the ethanol may be fed directly to one or more vessel suitable for chemical synthesis of ethylene. Similarly, in certain embodiments of the invention ethylene may be continuously recovered from the method and optionally fed directly to a chemical synthesis reaction for the production of another chemical product. In other embodiments, ethylene is converted or used in the production of other chemical products in situ on a continuous basis.

Microorganisms

Any one or more microorganisms capable of fermenting a substrate comprising CO to produce ethanol may be used in the present invention. By way of example only, microorganisms of the genus Moorella, Clostridia, Ruminococcus, Acetobacterium, Eubacterium, Butyribacterium, Oxobacter, Methanosarcina, Methanosarcina, and Desulfotomaculum may be used.

By way of example, in one embodiment, the one or more microorganism is of the genus Clostridium, including strains of Clostridium ljungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091), Clostridium ragsdalei (WO/2008/028055) and Clostridium autoethanogenum (Abrini et al, Archives of Microbiology 161: pp 345-351).

By way of further example, the one or more microorganism is Moorella sp HUC22-1, (Sakai et al, Biotechnology Letters 29: pp 1607-1612), or of the genus Carboxydothermus as described by Svetlichny, V. A., Sokolova, T. G. et al (1991) (Systematic and Applied Microbiology 14: 254-260), Moorella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, or Desulfotomaculum kuznetsovii (Simpa et. al. Critical Reviews in Biotechnology, 2006 Vol. 26. Pp 41-65). Other specific examples of carboxydotrophic anaerobic bacteria which may be used in the invention will be understood by a person of skill in the art.

In certain embodiments of the invention the one or more microorganisms used in the fermentation is Clostridium autoethanogenum. In certain embodiments the Clostridium autoethanogenum is a Clostridium autoethanogenum having the identifying characteristics of the strain deposited at the German Resource Centre for Biological Material (DSMZ) under the identifying deposit number DMS19630 or the strain deposited at the DSMZ under the identifying deposit number DMS23693. In another embodiment the Clostridium autoethanogenum is a Clostridium autoethanogenum DMS 10061 or DMS23693.

In other embodiments, the one or more microorganism used in the fermentation is Clostridium ljungdahlii or Clostridium ragsdalei. In certain embodiments the Clostridium ljungdahlii has the identifying characteristics of the strain deposited at the German Resource Centre for Biological Material (DSMZ) under the identifying deposit number DMS13582 and the Clostridium ragsdalei has the identifying characteristics of the strain deposited at the American Type Culture Collection (ATCC) under the identifying deposit number ATCC-BAA 622™, however it should be appreciated that other strains may be used.

It should be appreciated that the invention may be applied to a mixed culture of two or more bacteria.

Culturing of the bacteria used in the method of the invention may be conducted using any number of processes known in the art for culturing and fermenting substrates using anaerobic bacteria. Exemplary techniques are provided in the “Examples” section of this document. By way of further example, those processes generally described in the following articles using gaseous substrates for fermentation may be utilised: K. T. Klasson, M. D. Ackerson, E. C. Clausen and J. L. Gaddy (1991). Bioreactors for synthesis gas fermentations resources. Conservation and Recycling, 5; 145-165; K. T. Klasson, M. D. Ackerson, E. C. Clausen and J. L. Gaddy (1991). Bioreactor design for synthesis gas fermentations. Fuel. 70. 605-614; K. T. Klasson, M. D. Ackerson, E. C. Clausen and J. L. Gaddy (1992). Bioconversion of synthesis gas into liquid or gaseous fuels. Enzyme and Microbial Technology. 14; 602-608; J. L. Vega, G. M. Antorrena, E. C. Clausen and J. L. Gaddy (1989). Study of Gaseous Substrate Fermentation: Carbon Monoxide Conversion to Acetate. 2. Continuous Culture. Biotech. Bioeng. 34. 6. 785-793; J. L. Vega, E. C. Clausen and J. L. Gaddy (1989). Study of gaseous substrate fermentations: Carbon monoxide conversion to acetate. 1. Batch culture. Biotechnology and Bioengineering. 34. 6. 774-784; and, J. L. Vega, E. C. Clausen and J. L. Gaddy (1990). Design of Bioreactors for Coal Synthesis Gas Fermentations. Resources, Conservation and Recycling. 3. 149-160.

Substrates

A substrate comprising carbon monoxide, preferably a gaseous substrate comprising carbon monoxide, is used in the fermentation reaction to produce ethanol in the methods of the invention. The gaseous substrate may be a waste gas obtained as a by-product of an industrial process, or from some other source such as from combustion engine (for example automobile) exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production, coke manufacturing and methane reforming. In these embodiments, the CO-containing gas may be captured from the industrial process before it is emitted into the atmosphere, using any convenient method. Depending on the composition of the gaseous substrate comprising carbon monoxide, it may also be desirable to treat it to remove any undesired impurities, such as dust particles before introducing it to the fermentation. For example, the gaseous substrate may be filtered or scrubbed using known methods.

In other embodiments of the invention, the gaseous substrate comprising carbon monoxide may be sourced from the gasification of biomass. The process of gasification involves partial combustion of biomass in a restricted supply of air or oxygen. The resultant gas typically comprises mainly CO and H₂, with minimal volumes of CO₂, methane, ethylene and ethane. For example, biomass by-products obtained during the extraction and processing of foodstuffs such as sugar from sugarcane, or starch from maize or grains, or non-food biomass waste generated by the forestry industry may be gasified to produce a CO-containing gas suitable for use in the present invention.

The CO-containing substrate will typically contain a major proportion of CO, such as at least about 15% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

It is not necessary for the gaseous substrate to contain any hydrogen, however this is not considered detrimental to ethanol production. The gaseous substrate may also contain some CO₂ for example, such as about 1% to about 80% by volume, or 1% to about 30% by volume. In one embodiment it contains about 5% to about 10% by volume. In another embodiment the gaseous substrate contains approximately 20% CO₂ by volume.

Typically, the carbon monoxide will be added to the fermentation reaction in a gaseous state. However, the invention should not be considered to be limited to addition of the substrate in this state. For example, the carbon monoxide could be provided in a liquid. For example, a liquid may be saturated with a carbon monoxide containing gas and then that liquid added to a bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used.

In one embodiment of the invention, a combination of two or more different substrates may be used in the fermentation reaction.

In addition, it is often desirable to increase the CO concentration of a substrate stream (or CO partial pressure in a gaseous substrate) and thus increase the efficiency of fermentation reactions where CO is a substrate. Increasing CO partial pressure in a gaseous substrate increases CO mass transfer into a fermentation media. The composition of gas streams used to feed a fermentation reaction can have a significant impact on the efficiency and/or costs of that reaction. For example, O2 may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

Media

It will be appreciated that for growth of the one or more microorganisms and substrate to ethanol fermentation to occur, in addition to the substrate, a suitable nutrient medium will need to be fed to the bioreactor. A nutrient medium will contain components, such as vitamins and minerals, sufficient to permit growth of the micro-organism used. Anaerobic media suitable for the fermentation of ethanol using CO as the sole carbon source are known in the art. For example, suitable media are described in U.S. Pat. Nos. 5,173,429 and 5,593,886 and WO 02/08438, WO2007/115157, WO2008/115080 and WO2009/022925. By way of further example only, anaerobic media suitable for the growth of Clostridium autoethanogenum are known in the art, as described for example by Abrini et al (Clostridium autoethanogenum, sp. November, An Anaerobic Bacterium That Produces Ethanol From Carbon Monoxide; Arch. Microbiol., 161: 345-351 (1994)). The “Examples” section herein after provides further examples of suitable media.

Fermentation Conditions

The fermentation should desirably be carried out under appropriate conditions for the substrate to ethanol fermentation to occur. Reaction conditions that should be considered include temperature, media flow rate, pH, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

The optimum reaction conditions will depend partly on the particular microorganism of used. However, in general, it is preferred that the fermentation be performed at a pressure higher than ambient pressure. Operating at increased pressures allows a significant increase in the rate of CO transfer from the gas phase to the liquid phase where it can be taken up by the micro-organism as a carbon source for the production of ethanol. This in turn means that the retention time (defined as the liquid volume in the bioreactor divided by the input gas flow rate) can be reduced when bioreactors are maintained at elevated pressure rather than atmospheric pressure.

Also, since a given CO-to-ethanol conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-product fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the ethanol product is consumed by the culture.

Examples of fermentation conditions suitable for anaerobic fermentation of a substrate comprising CO are detailed in WO2007/117157, WO2008/115080, WO2009/022925 and WO02/08438. It is recognised the fermentation conditions reported therein can be readily modified in accordance with the methods of the instant invention.

Bioreactor

Fermentation reactions may be carried out in any suitable bioreactor as described previously herein. In some embodiments of the invention, the bioreactor may comprise a first, growth reactor in which the micro-organisms are cultured, and a second, fermentation reactor, to which broth from the growth reactor is fed and in which most of the fermentation product (ethanol, for example) is produced.

Product Recovery

The fermentation will result in a fermentation broth comprising a desirable product (ethanol) and/or one or more by-products (such as acetate and butyrate) as well as bacterial cells, in a nutrient medium.

In certain embodiments the ethanol produced in the fermentation reaction is converted to ethylene directly from the fermentation broth. In other embodiments, the ethanol is first recovered from the fermentation broth before conversion to ethylene.

In certain embodiments, the recovery of ethanol comprises continuously removing a portion of broth and recovering ethanol from the removed portion of the broth.

In particular embodiments the recovery of ethanol includes passing the removed portion of the broth containing ethanol through a separation unit to separate bacterial cells from the broth, to produce a cell-free ethanol-containing permeate, and returning the bacterial cells to the bioreactor. The cell-free ethanol-containing permeate may then be used for subsequent conversion to ethylene.

In certain embodiments, the recovering of ethanol and/or one or more other products or by-products produced in the fermentation reaction comprises continuously removing a portion of the broth and recovering separately ethanol and one or more other products from the removed portion of the broth.

In some embodiments the recovery of ethanol and/or one or more other products includes passing the removed portion of the broth containing ethanol and/or one or more other products through a separation unit to separate bacterial cells from the ethanol and/or one or more other products, to produce a cell-free ethanol-and one or more other product-containing permeate, and returning the bacterial cells to the bioreactor.

In the above embodiments, the recovery of ethanol and one or more other products preferably includes first removing ethanol from the cell-free permeate followed by removing the one or more other products from the cell-free permeate. Preferably the cell-free permeate is then returned to the bioreactor.

Ethanol, or a mixed product stream containing ethanol, may be recovered from the fermentation broth by methods known in the art. Exemplary methods include those described in WO07/117157, WO08/115080, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example only, ethanol may be recovered from the fermentation broth using methods such as fractional distillation or evaporation, pervaporation, and extractive fermentation. Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

By-products such as acids including acetate and butyrate may also be recovered from the fermentation broth using methods known in the art. For example, an adsorption system involving an activated charcoal filter or electrodialysis may be used.

In the case of use of an activated charcoal filter, it is preferred that microbial cells are first removed from the fermentation broth using a suitable separation unit. Numerous filtration-based methods of generating a cell free fermentation broth for product recovery are known in the art. The cell free ethanol—and acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used in the processes of the present invention. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

In certain embodiments of the invention, ethanol and by-products are recovered from the fermentation broth by continuously removing a portion of the broth from the bioreactor, separating microbial cells from the broth (conveniently by filtration, for example), and recovering ethanol and optionally other alcohols and acids from the broth. Alcohols may conveniently be recovered for example by distillation, and acids may be recovered for example by adsorption on activated charcoal. The separated microbial cells are preferably returned to the fermentation bioreactor. The cell free permeate remaining after the alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted during recovery of ethanol and/or other products or by-products, the pH should be re-adjusted to a similar pH to that of the broth in the fermentation bioreactor, before being returned to the bioreactor.

In certain embodiments, the ethanol is continuously recovered from the fermentation broth or bioreactor and fed directly for chemical conversion to ethylene. For example, the ethanol may be fed directly through a conduit to one or more vessel suitable for chemical synthesis of ethylene or other down stream chemical products.

Conversion to Other Chemical Products

A number of known methods may be used for the production of ethylene from ethanol. For example, previously reported catalysts for the dehydration of ethanol include activated clay, phosphoric acid, sulphuric acid, activated alumina, transition metal oxide, transition metal composite oxide, heteropolyacid and zeolites. Typically, catalysts used in current industrial dehydrations of ethanol are based on activated alumina systems. By way of non-limiting example, Syndol (with a main composition of Al₂O₃—MgO/SiO₂) has been commercially used to dehydrate ethanol for over 20 years. Syndol can be used to dehydrate anhydrous ethanol, or partially hydrated ethanol, such as 95% ethanol, to produce ethylene In such a process, ethanol is typically passed over the catalyst at temperatures in excess of 300° C. to give the olefin with conversion rates and selectivity's exceeding 95%. Other zeolite based catalysts incude TiO₂/4 Å Al₂O₃ zeolite.

In certain embodiments, the ethanol is heated with an excess of concentrated sulphuric acid at a temperature of 170° C. The gases produced are the passed over a sodium hydroxide solution to remove carbon dioxide and sulphur dioxide. The ethylene is collected over water. The stoichiometry of the reaction is as follows;

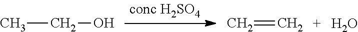

In alternative embodiments, the catalyst used is concentrated phosphoric acid.

In certain embodiments, the ethanol is passed over a heated aluminium oxide powder to produce ethylene and water vapour according to the following stoichiometry;

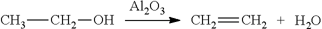

In certain embodiments ethanol is provided to a vessel. The ethanol is boiled and the resulting ethanol vapour is passed over an aluminium oxide catalyst, over heat. The ethanol vapour is converted to ethylene and water vapour according to the above stoichiometry.

Ethylene can subsequently be used in a variety of processes for producing commercially useful chemical products.

Ethylene is a high value gaseous compound which is widely used in industry. By way of example, ethylene may be used as an anaesthetic or as a fruit ripening agent, as well as in the production of a number of other chemical products. By way of example, ethylene may be used to produce polyethylene and other polymers, such as polystyrene, ethylene oxide, ethylene dichloride, ethylene dibromide, ethyl chloride and ethylbenzene. Ethylene oxide is, for example, a key raw material in the production of surfactants and detergents and in the production of ethylene glycol, which is used in the automotive industry as an antifreeze product. Ethylene dichloride, ethylene dibromide, and ethyl chloride may be used to produce products such as polyvinyl chloride, trichloroethylene, perchloroethylene, methyl chloroform, polyvinylidiene chloride and copolymers, and ethyl bromide. Ethylbenzene is a precursor to styrene, which is used in the production of polystyrene (used as an insulation product) and styrene-butadiene (which is rubber suitable for use in tires and footwear).

It should be appreciated that the methods of the invention may be integrated or linked with one or more methods for the production of downstream chemical products from ethylene. For example, the methods of the invention may feed ethylene directly or indirectly to chemical processes or reactions sufficient for the conversion or production of other useful chemical products. In some embodiments, as noted herein before, ethanol is converted to one or more chemical products directly via the intermediate compound ethylene without the need for recovery of ethylene from the method before subsequent use in production of the one or more chemical products.

In particular embodiments, ethanol is converted to ethylene by one or more chemical processes, which in turn is converted to one or more chemical products by one or more chemical processes. In particular embodiments, the one or more chemical products are produced without recovering the ethylene. In another embodiment, ethanol is converted to one or more chemical products in a single chemical process via the ethylene intermediate compound.

The invention will now be described in more detail with reference to the following non-limiting examples.

### EXAMPLES

### Example 1

Materials and Methods:

Solution A

| | | | |
|---|---|---|---|
| NH₄Ac | 3.083 g | KCl | 0.15 g |
| MgCl₂•6H₂O | 0.4 g | NaCl (optional) | 0.12 g |
| CaCl₂•2H₂O | 0.294 g | Distilled Water | Up to 1 L |

Solution B

| Biotin | 20.0 mg | Calcium D-(*)- | 50.0 mg |
|---|---|---|---|
| | | pantothenate | |
| Folic acid | 20.0 mg | Vitamin B12 | 50.0 mg |
| Pyridoxine•HCl | 10.0 mg | p-Aminobenzoic | 50.0 mg |
| | | acid | |
| Thiamine•HCl | 50.0 mg | Thioctic acid | 50.0 mg |
| Riboflavin | 50.0 mg | Distilled water | To 1 Litre |
| Nicotinic acid | 50.0 mg | | |
| Component | mmol/L H2O | Component | mmol/L H2O |

Solution C

| | | | |
|---|---|---|---|
| FeCl₃ | 0.1 | Na₂SeO₃ | 0.01 |
| CoCl₂ | 0.05 | Na₂MoO₄ | 0.01 |
| NiCl₂ | 0.05 | ZnCl₂ | 0.01 |
| H₃BO₃ | 0.01 | MnCl2 | 0.01 |
| | | Na2WO3 | 0.01 |

Preparation of Cr (II) solution: A 1 L three necked flask was fitted with a gas tight inlet and outlet to allow working under inert gas and subsequent transfer of the desired product into a suitable storage flask. The flask was charged with CrCl₃.6H₂O (40 g, 0.15 mol), zinc granules [20 mesh] (18.3 g, 0.28 mol), mercury (13.55 g, 1 mL, 0.0676 mol) and 500 mL of distilled water. Following flushing with N₂ for one hour, the mixture was warmed to about 80° C. to initiate the reaction. Following two hours of stirring under a constant N₂ flow, the mixture was cooled to room temperature and continuously stirred for another 48 hours by which time the reaction mixture had turned to a deep blue solution. The solution was transferred into N₂ purged serum bottles and stored in the fridge for future use.

Bacteria: Two types of Clostridium autoethanogenum were used in the following examples. DSM 19630 and DSM 23693, both deposited at the German Resource Centre for Biological Material (DSMZ).

Sampling and analytical procedures: Media samples were taken from the CSTR reactor at intervals over the course of each fermentation. Each time the media was sampled care was taken to ensure that no gas was allowed to enter into or escape from the reactor.

HPLC: HPLC System Agilent 1100 Series. Mobile Phase: 0.0025N Sulfuric Acid. Flow and pressure: 0.800 mL/min. Column: Alltech IOA; Catalog # 9648, 150×6.5 mm, particle size 5 μm. Temperature of column: 60° C. Detector: Refractive Index. Temperature of detector: 45° C.

Method for sample preparation: 400 μL of sample and 50 μL of 0.15M ZnSO₄ and 50 μL of 0.15M Ba(OH)₂ are loaded into an Eppendorf tube. The tubes are centrifuged for 10 min. at 12,000rpm, 4° C. 200 μL of the supernatant are transferred into an HPLC vial, and 5 μL are injected into the HPLC instrument.

Headspace Analysis: Measurements were carried out on a Varian CP-4900 micro GC with two installed channels. Channel 1 was a 10 m Mol-sieve column running at 70° C., 200 kPa argon and a backflush time of 4.2 s, while channel 2 was a 10 m PPQ column running at 90° C., 150 kPa helium and no backflush. The injector temperature for both channels was 70° C. Runtimes were set to 120 s, but all peaks of interest would usually elute before 100 s.

Cell Density: Cell density was determined by counting bacterial cells in a defined aliquot of fermentation broth. Alternatively, the absorbance of the samples was measured at 600nm (spectrophotometer) and the dry mass determined via calculation according to published procedures.

A: Batch Fermentation in CSTR

Approximately 1500 mL of solution A was transferred into a 1.5 L fermenter and sparged with nitrogen. Resazurin (1.5 mL of a 2 g/L solution) and H₃PO₄ (85% solution, 2.25 mL) was added and the pH adjusted to 5.3 using concentrated NH₄OH(aq). Nitrilotriacetic acid (0.3 ml of a 0.15M solution) was added prior to 1.5 ml of solution C. This was followed by NiCl2 (0.75 ml of 0.1M solution) and Na₂WO₃ (1.5 mL of a 0.01M solution). 15ml of solution B was added and the solution sparged with N2 before switching to CO containing gas (50% CO; 28% N2, 2%H2, 20% CO2) at 70 mL/min. The fermenter was then inoculated with 200 ml of a Clostridium autoethanogenum 19630 culture. The fermenter was maintained at 37° C. and stirred at 300 rpm. During this experiment, Na2S solution (0.2M solution) was added at a rate of approx 0.3 ml/hour. Substrate supply was increased in response to the requirements of the microbial culture.

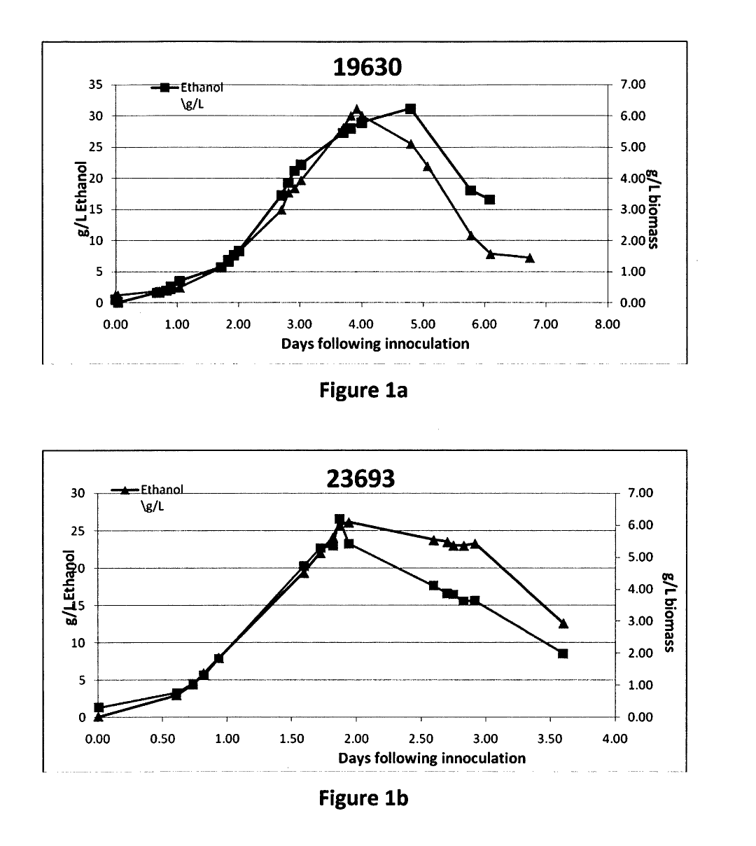

FIG. 1a illustrates ethanol production by the bacteria.

B: Batch Fermentation in CSTR

Approximately 1500 mL of solution A was transferred into a 1.5 L fermenter and sparged with nitrogen. Resazurin (1.5 mL of a 2 g/L solution) and H₃PO₄ (85% solution, 2.25 mL) was added and the pH adjusted to 5.3 using concentrated NH₄OH(aq). Nitrilotriacetic acid (0.3 ml of a 0.15M solution) was added prior to 1.5 ml of solution C. Na₂WO₃ (1.5 mL of a 0.01M solution) was added. 15 ml of Solution B was added and the solution sparged with N2 before switching to CO containing gas (50% CO; 50% N2) at 60 mL/min. The fermenter was then inoculated with 180 ml of a Clostridium autoethanogenum 23693 culture. The fermenter was maintained at 37° C. and stirred at 300 rpm. During this experiment, Na2S solution (0.5M solution) was added at a rate of approx 0.12 ml/hour. Substrate supply was increased in response to the requirements of the microbial culture.

FIG. 1b illustrates ethanol production by the bacteria.

### Example 2

Materials and Methods:

Bacterial strains and growth conditions: C. autoethanogenum DSM 10061 and C. ljungdahlii DSM 13582 were obtained from DSMZ (Deutsche Sammlung von Mikroorganismen and Zellkulturen GmbH) and C. ragsdalei ATCC-BAA 622TM from ATCC (American Type Culture Collection). All organisms were cultivated anaerobically in modified PETC medium (ATCC medium 1754) at 30° C. (C. ragsdalei) or respectively 37° C. (C. autoethanogenum and C. ljungdahlii).

The modified PETC medium contained (per L) 1 g NH4Cl, 0.4 g KCl, 0.2 g MgSO4×7 H2O, 0.8 g NaCl, 0.1 g KH2PO4, 20 mg CaCl2×2 H2O, 10 ml trace elements solution (see below), 10 ml Wolfe's vitamin solution (see below), 2 g NaHCO3, and 1 mg resazurin. After the pH was adjusted to 5.6, the medium was boiled, dispensed anaerobically, and autoclaved at 121° C. for 15 min. Steel mill waste gas (composition: 44% CO, 32% N2, 22% CO2, 2% H2) collected from a New Zealand steel site in Glenbrook, NZ or 0.5% (w/v) fructose (with N2 headspace) were used as carbon source. The media had a final pH of 5.9 and was reduced with Cystein-HCI and Na2S in a concentration of 0.008% (w/v).

The trace elements solution consisted of 2 g nitrilotriacetic acid (adjusted to pH 6 with KOH before addition of the remaining ingredients), 1 g MnSO4, 0.8 g Fe(SO4)2(NH4)2×6 H2O, 0.2 g CoCl2×6 H2O, 0.2 mg ZnSO4×7 H2O, 20 mg CuCl2×2 H2O, 20 mg NiCl2×6 H2O, 20 mg Na2MoO4×2 H2O, 20 mg Na2SeO4, and 20 mg Na2WO4 per liter.

Wolfe's vitamin solution (Wolin, E. A., Wolin, M. J. & Wolfe, R. S. Formation of methane by bacterial extracts. J. Biol. Chem. 238, 2882-2886 (1963)) contained (per L) 2 mg biotin, 2 mg folic acid, 10 mg pyridoxine hydrochloride, 5 mg thiamine-HCl, 5 mg riboflavin, 5 mg nicotinic acid, 5 mg calcium D-(+)-pantothenate, 0.1 mg vitamin B12, 5 mg p-aminobenzoic acid, and 5 mg thioctic acid.

Batch Fermentation in Serum bottles

Growth experiments were carried out in a volume of 100 ml PETC media in plastic-coated 500-ml-Schott Duran® GL45 bottles with butyl rubber stoppers and 200 kPa steel mill waste gas as sole energy and carbon source. Growth was monitored by measuring the optical density at 600 nm (OD600 nm) and metabolic end products were analyzed by HPLC.

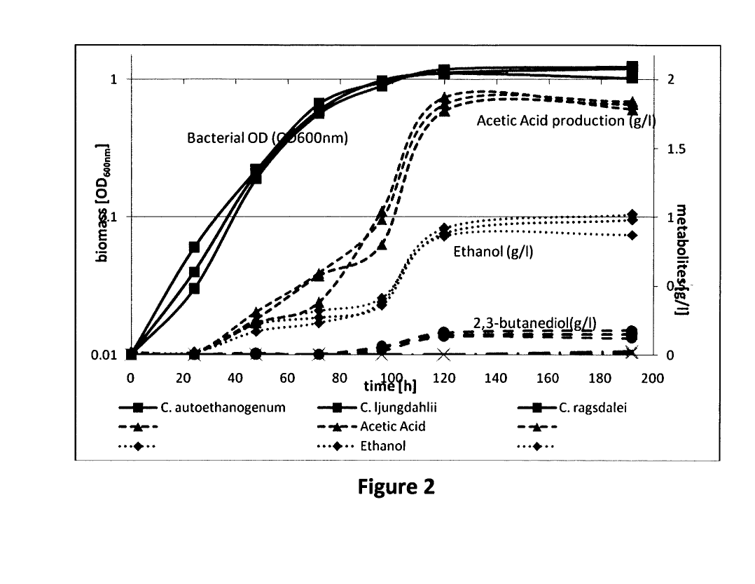

FIG. 2 illustrates ethanol production by the bacteria.

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in the United States of America or any other country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1 to 19. (canceled)

20. A method for producing one or more chemical products, the method comprising;
a. flowing a gaseous substrate comprising CO into a bioreactor containing a culture of one or more microorganisms; and
b. anaerobically fermenting the substrate comprising CO to produce ethanol; and
c. converting the ethanol produced in step (b) to one or more products via the intermediate compound ethylene.

21. The method of claim 20 wherein the ethanol is recovered before it is converted to ethylene in step (c).

22. The method according to claim 20 wherein the ethylene is recovered before it converted to one or more chemical products in step (c)

23. The method of claim 20 wherein the ethanol is converted to one or more products without recovery of ethylene during step (c).

24. The method of claim 20 wherein the substrate comprises at least 20% CO by volume, to about 95% CO by volume.

25. The method of claim 24 wherein the substrate comprises at least 40% CO by volume.

26. The method of claim 20 wherein the concentration of ethanol produced by the fermentation is at least 10 g/L.

27. The method of claim 20 wherein the microorganism is selected from the group consisting of Clostridium autoethanogenum, Clostridium ragsdalei, Clostridium ljungdahlii and mixtures thereof.

28. The method of claim 27 wherein the microorganism is Clostridium autoethanogenum strain deposited at the German Resource Centre for Biological Material (DSMZ) under the identifying deposit number DSM 23693.

29. The method of claim 20 wherein the CO containing substrate is an industrial waste gas obtained as a by-product of an industrial process.

30. The method of claim 29 wherein the industrial process is selected from the group consisting of ferrous metal products manufacturing, steel manufacturing, non-ferrous products manufacturing, petroleum refining processes, gasification of coal, electric power production, carbon black production, ammonia production, methanol production, coke manufacturing and methane reforming.

31. The method of claim 20 where the products produced from ethylene are selected from the group consisting of polyethylene, polystyrene, ethylene oxide, ethylene dichloride, ethylene dibromide, ethyl chloride, ethylebenzene and mixtures thereof.

32. A method for producing ethylene, the method comprising;
a. flowing a gaseous substrate comprising CO into a bioreactor containing a culture of one or more microorganisms;
b. anaerobically fermenting the substrate of step (a) to produce ethanol; and
c. converting the ethanol produced in step (b) to ethylene.
